(19) United States
(12) Patent Application Publication
(10) Pub. No.: US 2009/0012418 A1
(43) Pub. Date: Jan. 8, 2009

(54) MEDICAL SURGICAL SPONGE AND INSTRUMENT DETECTION SYSTEM AND METHOD

(76) Inventor: Frank Gerlach, Mississauga (CA)

Correspondence Address: H. Dale Langley, Jr.;The Law Office of H.Dale Langley, Jr., 610 West Lynn, Austin, TX, 78703, US

(21) Appl. No.: 11/772,976

(22) Filed: Jul. 3, 2007

(51) Int. Cl. A61B 5/06

(52) U.S. Cl. 600/550

(57) ABSTRACT

A magnetic detector locates magnetically tagged items, such as surgical sponges or instruments, introduced to and remaining in the body cavity of a surgical patient. The magnetic detector is a wand or probe that is manually handleable, and spatially manipulable and moveable, by an operator, such as a medical professional. The detector is moved about at a spatial location of an ambient magnetic field at the patient and operating table. A characteristic of the ambient magnetic field at the spatial location is saved by the detector. The detector is subsequently moved about at the same spatial location. If any magnetically tagged item is present in the vicinity of the detector during this subsequent movement at the spatial location, an anomalous magnetic effect is detected by comparison of the prior detection reading absent the tagged item to the detection reading in presence of the tagged item in the patient. The anomalous magnetic effect, and thus the tagged item, is locatable spatially in the patient, by three-dimensional sensor(s), arrays of sensors, and pluralities of arrays of sensors of the detector. The different detection readings with and without presence of the anomalous magnetic effect caused by presence of the tagged item in the patient are calculable as scalar, vector array, and/or gradient array determinations, according to the particular number and configuration of sensors in the detector.

### BACKGROUND OF THE INVENTION

The present invention generally relates to detectors for medical and surgical applications and, more particularly, relates to detectors for indicating presence of any misplaced extraneous items, such as surgical sponges and instruments, in a patient's body cavity in medical procedures.

During medical procedures, such as surgeries and the like, surgical sponges, instruments, tools and other extraneous items are placed within incisions and spaces of the patient's body. These items must be removed from the body cavity prior to closing on completion of surgery. If the items are not removed, then there is great risk of infection and other adverse conditions in the patient. Doctors, nurses, and hospitals implement strict procedures and protocols to seek to assure recovery of the items prior to closing on completion of surgery.

Certain detection devices, mechanisms, and schemes have been available for seeking to find and locate any such extraneous items. Notwithstanding all the precautions taken in efforts to avoid the problems, the extraneous items in patients are misplaced and left in the body in a number of cases. Beyond the potential adverse medical consequences to the patient, liability concerns to hospitals and medical professionals are very significant. Malpractice insurers to physicians have particular concern about these problems.

Several conventional detection schemes require invasive procedures to the patient. These detectors work through physical invasion of the patient's body, either by tangible devices inserted into the patient or by radio, sonic, x-ray, electrical, or radiation waves or the like, directed to/through the body. Examples of these schemes include detectors of radio frequency identification (RFID) tags, electro- or magneto-mechanically stimulated resonator tags, or electrical, radiative or sound emitter tags. In x-ray scanning for extraneous items, the items are tagged with radio-opaque wire or other object detectable by x-ray radiology. The detection procedure, such as x-ray, is performed upon surgery procedure completion but prior to body close. X-ray detection, in particular, has been a typical technique for detection of extraneous items.

In these invasive detection schemes, the bodily invasions required in order to perform the detection steps is potentially adverse to the patient during surgery or recovery. Added foreign matters and structures, radiation, radio or magnetic resonance waves, stimulated vibrations of resonant tags, mechanically, electrical, or electromagnetic active tags within the patient and the like, must be introduced to and/or activated in the patient's body. These bodily invasions subject the patient to potential complications, for example, bleeding, sepsis, burn, internal irritation, surgery complications and other harmful conditions.

Other typical techniques include non-invasive labeling and/or counting procedures. Examples of the techniques include manual count, counting bins, manually reviewed check-lists, numbering systems, external electrical or light sensors for registering each item after removal, and other externally situated implements and machines that count items retrieved from the patient. Each extraneous item introduced to the patient's body is recorded/noted prior to introduction, then on removal, each item is accounted for prior to close of the surgical incision site. These non-invasive labeling and counting techniques are presently most commonly employed.

Generally in these non-invasive techniques, respective sponges, instruments, and other items for the surgery are identified prior to introduction in the patient's body cavity. The identification of the items has, at times, included labeling or marking. In certain instances, accounting for the items on removal from the body cavity is performed manually by medical personnel and/or by automated devices external to the body. For example, certain hoppers or bins having associated count mechanisms (e.g., IR, RFID or other sensors) that register each item successfully removed and placed in the bin.

Non-invasive schemes have merit to the extent that items are fully identified and counted. However, if either the count is inaccurate or items are missing, then search for the items is required. This requires physical re-invasion of the patient by one of the invasive techniques or by manual search within the body by the medical professional. Also, the non-invasive counts are typically subject to human errors, including either in making manual count, in adequately mechanically/automatedly registering the count, or in other respects.

In any event, the prior invasive and non-invasive techniques and procedures have been faulty in instances. The devices for the techniques and procedures have tended to be wieldy, complex and pricey. The conventional solutions, other than simple marking and manual counting, have not been widely adopted because of the inaccuracies and other issues. Additional problems have also been presented by the various conventional alternatives, particularly, such as the extensively invasive procedures that can be required. A viable, simpler, compact, and inexpensive solution is drastically needed.

It would, therefore, be a significant improvement in the art and technology to provide systems and methods and other improvements for finding extraneous items that may remain in the patient's body. Such improvements can yield significant medical, safety and therapeutic advantages and other benefits for the patient. The improvements also can aid in avoiding liabilities of medical professionals, hospitals and insurers because of misplaced and remaining items in the patient. Moreover, any such improvements that provide less wieldy, less invasive, simpler and relatively inexpensive advantages are highly desired solutions.

### SUMMARY OF THE INVENTION

An embodiment of the invention is a magnet detector for locating a magnetically tagged item in a surgical patient. The detector includes a three-dimensional (x,y,z) sensor, moveable throughout a magnetic field at the patient, a first output of the sensor represents a first-sensed magnetic field strength vectors for three dimensions at a first spatial location of the sensor and a second output of the sensor represents a second-sensed magnetic field strength vectors for three dimensions at a second spatial location of the sensor; a processor communicatively connected to the sensor, for receiving the first output and the second output and calculating a value from the first output and the second output representative, respectively, of an ambient scalar (Mᵥ(x)_{ambient}) of the first-sensed magnetic field strength vectors and the second-sensed magnetic field strength vectors; and a storage for the value. A magnetic field anomaly is subsequently induced to the magnetic field at the first spatial location and the second spatial location. The sensor is moved throughout the magnetic field of the patient, and signals a third output of the sensor that represents a third-sensed magnetic field strength vectors and a fourth-sensed magnetic field strength vectors for three dimensions at the first spatial location and the second spatial location, respectively, of the sensor. The processor receives the third output and the fourth output of the sensor, and calculates a different value from the third output and the fourth output, the different value representative, respectively, of a different anomalous scalar (Mᵥ(x)_{anomaly}) of the third-sensed magnetic field strength vectors and the fourth-sensed magnetic field strength vectors. If the different value is in excess of a threshold for the detector, the detector signals indicating presence and location of the magnetically tagged item.

Another embodiment of the magnet detector is for locating the magnetically tagged item. The detector includes a plurality of the three-dimensional (x,y,z) sensor maintained in an array, each of the plurality of sensors is communicatively connected to the processor. A respective first output of each of the plurality and a respective second output of each of the plurality are communicated to the processor. The respective first output and respective second output represent respective first-sensed magnetic field strength vectors at the first spatial location and second-sensed magnetic field strength vectors at the second spatial location for each respective sensor of the plurality of sensors. The processor receives each of the respective first output and the respective second output and calculates an ambient array value representative, respectively, of an ambient magnetic field strength array (Mᵥ(x,y)_{ambient}) at about the first spatial location and the second spatial location for each sensor of the plurality. The ambient magnetic field strength vector for each sensor of the plurality is saved in the storage. The magnetically tagged item is subsequently introduced and located at or about the first spatial location and the second spatial location of the magnetic field. The plurality of sensors are thereafter again moved throughout the magnetic field of the patient, and a representative third output and representative fourth output is signaled by each sensor to represent third-sensed and fourth-sensed magnetic field strength vectors for three dimensions at the first spatial location and the second spatial location, respectively, of the each sensor of the plurality. The processor receives the third output and the fourth output of each sensor, and calculates a difference array value from the third output and the fourth output. The difference array value represents, respectively, a different anomalous magnetic field strength array (Mᵥ(x,y)_{anomaly}) of the third-sensed magnetic field strength vector and the fourth-sensed magnetic field strength vector for each sensor of the plurality. If the different array value is in excess of a threshold for the detector, the detector signals to indicate presence and location of the magnetically tagged item.

Another embodiment of the magnet detector is for locating the magnetically tagged item. The detector includes a plurality of arrays of three-dimensional (x,y,z) sensors. Each array is respectively positioned as to a third perpendicular dimension (z) to the two dimensions (x,y). Each array is moveable throughout the magnetic field of the patient, and a first respective output of each respective array of the plurality signals a first-sensed magnetic field strength gradient for three dimensions at the first respective spatial location of each respective array of the plurality and a second respective output of each respective array of the plurality represents a second-sensed magnetic field strength gradient for three dimensions at the second respective spatial location of each respective array of the plurality. A processor receives each of the first respective output and each of the second respective output and calculates an ambient field gradient array value representative, respectively, of an ambient magnetic field strength gradient (Mᵥ(x,y,z)_{ambient}) at about the first spatial location and the second spatial location for each array of the plurality. The ambient field gradient array value for each array of the plurality, and each of the plurality of sensors of each array, is saved in a storage. A magnetic field anomaly, effected by presence of the magnetically tagged item in the magnetic field, is subsequently introduced to the magnetic field at the first spatial location and the second spatial location. The arrays are thereafter moved throughout the magnetic field of the patient, and a third representative output of each array is signaled representing a third-sensed magnetic field strength gradient and a fourth-sensed magnetic field strength gradient for three dimensions at the first spatial location and the second spatial location, respectively, of the each array. The processor receives the third output and the fourth output of each array, calculates a difference gradient array value from the third output and the fourth output. The difference gradient array value represents, respectively, a different anomalous magnetic field strength gradient array (Mᵥ(x,y,z)_{anomaly}) of the third-sensed magnetic field strength gradient and the fourth-sensed magnetic field strength gradient for each array. If the different gradient array value is in excess of a threshold for the detector, the detector signals indicating presence and location of the magnetically tagged item.

Yet another embodiment of the invention is a hand-holdable probe or wand incorporating at least one of the foregoing detectors.

Another embodiment of the invention is a method of detecting presence of a magnetic tag of an item in a surgical patient. The method includes first sensing a one-dimensional characteristic of a three-dimensional magnetic field at the patient at a spatial location; first processing the one-dimensional characteristic from the step of first sensing, to obtain an ambient magnetic field scalar (Mᵥ(x)_{ambient}) for the spatial location; second sensing the one-dimensional characteristic of the three-dimensional magnetic field at the patient at the spatial location, the magnetic tag present at about the spatial location; second processing the one-dimensional characteristic of the step of second sensing, to obtain an anomalous magnetic field scalar (Mᵥ(x)_{anomaly}) for the spatial location; calculating a difference value (Δ=|(Mᵥ(x)_{ambient})−(Mᵥ(x)_{anomaly})|) in the ambient magnetic field scalar and the anomalous magnetic field scalar for the spatial location; and signaling if the difference value exceeds a threshold level, indicative of presence of the magnetic tag of the device at about the spatial location.

Another embodiment of the invention is a method of detecting presence of a magnetic tag of an item in a surgical patient. The method includes first sensing a two-dimensional characteristic of a three-dimensional magnetic field at the patient at a spatial location, first processing the two-dimensional characteristic from the step of first sensing, to obtain an ambient magnetic field strength array (Mᵥ(x,y)_{ambient}) for the spatial location, second sensing the two-dimensional characteristic of the three-dimensional magnetic field at the patient at the spatial location, the magnetic tag present at about the spatial location, second processing the two-dimensional characteristic of the step of second sensing, to obtain an anomalous magnetic field strength array (Mᵥ(x,y)_{anomaly}) for the spatial location, calculating a difference array value (Δ=|(Mᵥ(x,y)_{ambient})−(Mᵥ(x,y)_{anomaly})|) in the ambient magnetic field strength array and the anomalous magnetic field strength array for the spatial location, and signaling if the difference array value exceeds a threshold level, indicative of presence of the magnetic tag of the device at about the spatial location.

Yet another embodiment of the invention is a method of detecting presence of a magnetic tag of an item in a surgical patient. The method includes first moving a detector at a spatial location of an ambient magnetic field at the patient, saving a characteristic of the ambient magnetic field at the spatial location, second moving the detector in a magnetic anomaly to the ambient magnetic field, induced to the ambient magnetic field at the spatial location the patient, and determining the presence of the magnetic anomaly at about the spatial location by the detector.

Another embodiment of the invention is a magnetic detector for locating a magnetically tagged item in a surgical patient. The item is contained within a patient body cavity located a distance along a one-dimensional axis in space from the magnetic detector. The detector includes a first sensor array in a first plane in space centered at and perpendicular to the one-dimensional, a second sensor array positioned in a second plane in space, parallel to the first plane but not in the first plane, centered at and perpendicular to the one-dimensional axis, a third sensor array positioned in a third plane in space, parallel to the first plane and second plane but not in either the first plane or the second plane, centered at and perpendicular to the one-dimensional axis. The magnetic detector has a three-dimensional volume of interest for detections. The three-dimensional volume extends in space along the one-dimensional axis generally into the body cavity in space. The three-dimensional volume is defined in space extending along the axis as centrum of the three-dimensional volume in direction of the axis. The volume has an outer extent defined by the greatest extension point in space from the axis that is outwardly extending for the first array in the first plane, the second array in the second plane, and the third array in the third plane, whichever is greatest outwardly extending in respective planes. The magnetic detector also has a sensor volume of interest for detections. The sensor volume extends in space three-dimensionally along the one-dimensional axis within the three-dimensional volume, and extends in direction of the axis having centrum along the axis. The sensor volume has an outer extent defined by a uniquely detectable region central to the three-dimensional volume for, collectively, the first array, the second array and the third array. The magnetic detector is capable of cancelling out any relevant ambient magnetic effects, automatically. The magnetic detector, when positioned at a location from the patient, finds the item at a vicinity of the patient body cavity centered at the axis extending into the patient, because of difference of magnetic vector gradient then-determined by the magnetic detector as being in excess of a threshold value, the threshold value being related to magnetic vector gradient for the same location as if without presence of the item.

Another embodiment of the invention is a detector for locating a magnetized tag in a human body. The magnetized tag is positioned in a direction (A) from the detector. The detector includes a first array of planarly arranged individual sensor elements having a first centrum located in the direction (A) with respect to the tag, of a first plane, and a second array of planarly arranged individual sensor elements having a second centrum located in the direction (A) with respect to the tag, of a second plane that is not the first plane. The second array is positioned, in space, parallel to the first array. An imaginary three-dimensional volume, extending longitudinally perpendicular to the first array and the second array, defined by extents of the first array and the second array in the first plane and respectively, second plane, forms an area of interest for detections by the detector. The detector effectively discounts, automatically, any relevant ambient magnetic effects substantially within the area of interest. The detector locates the tag in the human body when the tag is within the area of interest the tag, based on a difference value of magnetic vector gradient detected in presence of the tag versus magnetic vector gradient detected without pesence of the tag, at about the same location for the area of interest, respectively.

### BRIEF DESCRIPTION OF THE DRAWINGS

The present invention is illustrated by way of example and not limitation in the accompanying figures, in which like references indicate similar elements, and in which:

FIG. 1 illustrates a side view of a wand and a probe for detecting a magnetically tagged item, such as a surgical sponge or instrument, in a body cavity of a patient in surgery, according to certain embodiments of the invention;

FIG. 2 illustrates a top downward view of the wand and the probe of FIG. 1, in the body cavity of the patient, according to certain embodiments of the invention;

FIG. 3 illustrates a side and top perspective view of the wand and the probe of FIGS. 1 and 2, in the body cavity of the patient, according to certain embodiments of the invention;

FIGS. 4A-D illustrate, respectively, a top view of the wand, a bottom view of the wand, a side view of the wand, and a top and side perspective view of the wand of FIGS. 1-3, according to certain embodiments of the invention;

FIGS. 5A-B illustrate exemplary magnetic fields in vicinity of the patient and operating table of FIGS. 1-3, respectively, without presence in the patient of any tagged item and with induced magnetic anomaly caused by presence of the tagged item in the patient, according to certain embodiments of the invention;

FIGS. 6A-C illustrate, respectively, a single sensor for detection of three dimensions (x,y,z), a one-dimensional array of the sensors, and a two-dimensional array of the sensors, such as included as an operative sensor portion of the wand or the probe of FIGS. 1-3, according to certain embodiments of the invention;

FIGS. 7A-C illustrate a three-dimensional array of sensors, respectively, in a top downward view, in a side view, and in a top and side perspective view, such as included as a disc-shaped operative portion of the wand of FIGS. 1-3, according to certain embodiments of the invention;

FIG. 8 illustrates component embodiments of the wand of FIGS. 1-3 having the array of sensors of FIGS. 7A-C, according to certain embodiments of the invention;

FIG. 9 illustrates a method of detection scanning using the wand or the probe of other Figures, according to certain embodiments of the invention;

FIG. 10 illustrates a perspective side view of the probe of FIGS. 1-3, having the single sensor or the one- or two-dimensional array of sensors included in an operative sensing portion, according to certain embodiments of the invention;

FIG. 11 illustrates component embodiments of the probe of FIGS. 1-3, 10, according to certain embodiments of the invention;

FIGS. 12A-H illustrate various embodiments of magnetic tags affixed to or with surgical sponges (FIGS. 12A-F) and to surgical instruments (FIGS. 12G-H), according to certain embodiments of the invention;

FIG. 13 illustrates a system for counting surgical items on removal from the patient and disposal, and alternately, on retrieval of the items for use in the patient during surgery, according to certain embodiments of the invention; and

FIG. 14 illustrates an alternative embodiment of the system of FIG. 13, according to certain embodiments of the invention.

Certain of the Figures include x, y and z spatial dimension indicators. In such Figures, the three axes indicators (i.e., x, y and z) mean three dimensional space and the two axes indicators (i.e., x and y, y and z, and x and z) mean two dimensional space. In general in these Figures, the z-axis is vertical, the x-axis is longitudinal, and the y-axis is width; however, it is to be understood that these spatial orientations so indicated are not exclusive and are merely intended for representation and understanding in certain embodiments particularly described.

### DETAILED DESCRIPTION

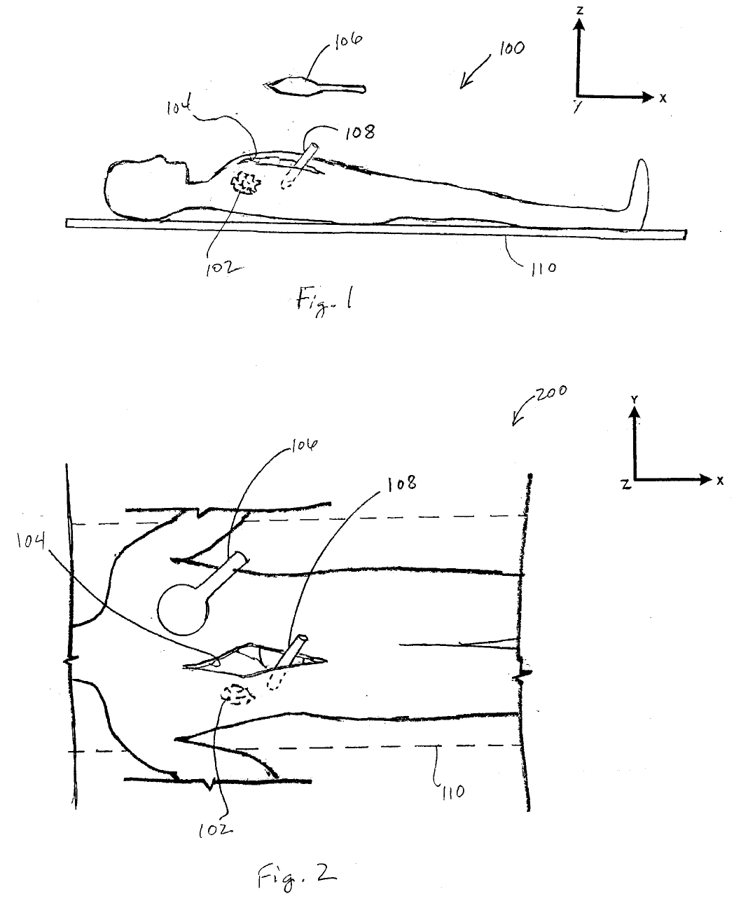

Referring to FIG. 1, a system 100 for detecting an item 102 contained within a cavity (e.g., a body cavity formed by an incision of a surgical patient 104 in FIG. 1 for reference purposes) includes a detector wand 106. The wand 106 is capable of detecting presence of the item within the patient 104. Alternately or additionally, the system 100 includes a detector probe 108. The probe 108 is also capable of detecting presence of the item with the patient 104. The wand 106 operates without intrusion into the body cavity of the patient 104, by multi-dimensional movement of the wand 106 (as hereinafter discussed). The probe 108 inserts into the body cavity of the patient 104 and is moveable within the patient 104 in multi-dimensions. The patient 104 is shown on an operating table 110, or the like, for reference purposes in FIG. 1.

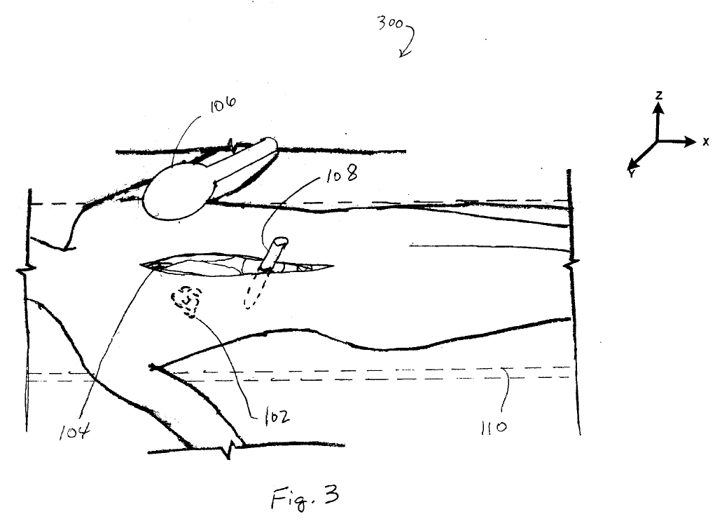

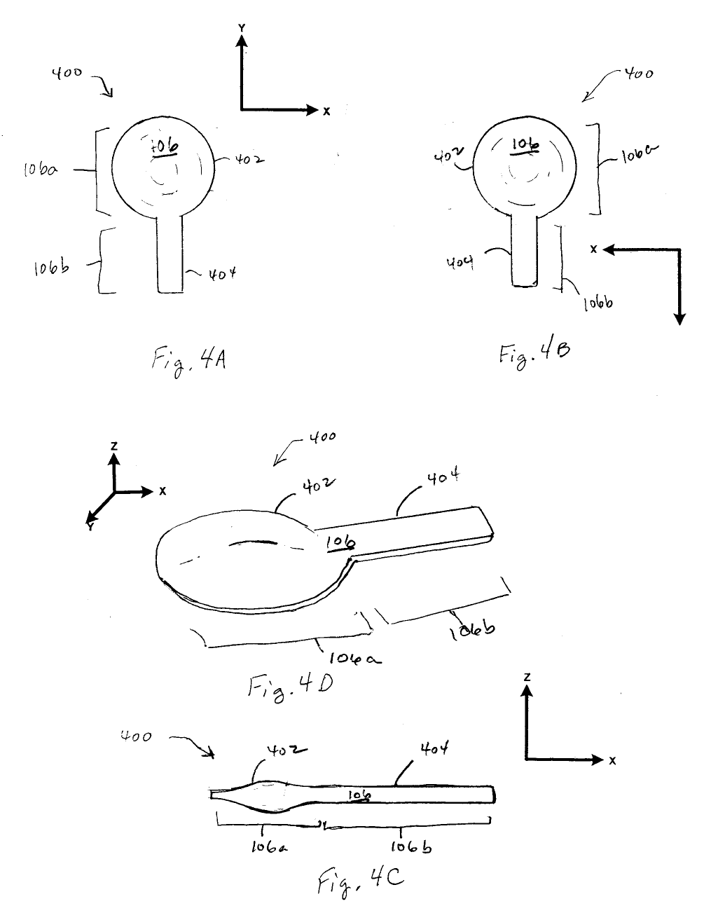

Referring to FIG. 2, in conjunction with FIG. 1, an above downward view 200 shows the patient 104 of FIG. 1 and the system 100. The system 100 includes the wand 106 positioned over the patient 104, such as by hand maneuvering by a medical professional (not shown in FIG. 2). The wand 106 is moveable, with respect to the patient 104, in all dimensions, including up and down (i.e., “z-axis”, vertical towards and from the patient in FIG. 2 and shown in FIGS. 1 and 3), left and right (i.e., “y-axis”, horizontal width across the body, left-side to ride-side, of the patient 104 in FIG. 1 and shown in FIGS. 2 and 3) and top and bottom (i.e., “x-axis”, horizontal longitude along the body, head-to-toe, of the patient 104 in FIGS. 1-3). The system 100, as mentioned, alternately or additionally includes the probe 108. The probe 108 is similarly movable by hand, with a sensing portion (later described in detail) at least partially disposed within the patient 104 through the surgical incision.

Referring to FIG. 3, in conjunction with FIGS. 1 and 2, a side and top perspective view 300 shows the patient of FIG. 1 and the system 100. The system 100 includes the wand 106 positioned vertically above the patient 104 in the body portion of interest for detection of the item 102. The alternate or additional feature of the probe 108 of the system 100 is partially residing in the body cavity of the patient 104 through the surgical incision. Each of the wand 106 and the probe 108 are capable of detecting presence of the item 102 lodged in the patient 104.

### Wand Detector:

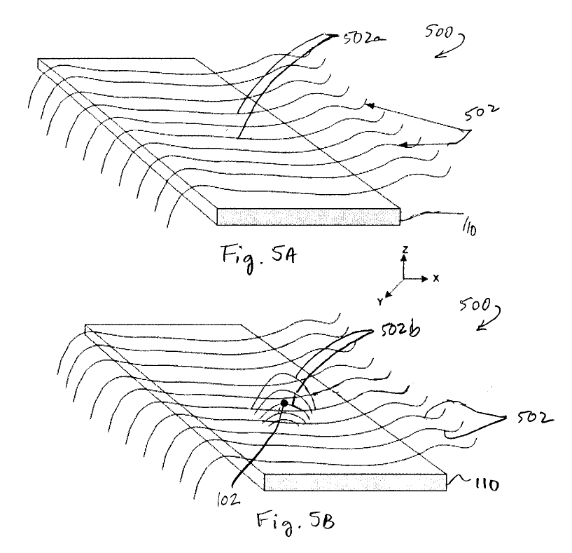

Referring to FIGS. 4A-4D, a detector device 400 for locating presence of the item 102, for example, the wand 106, has an operative detector 106a and a handle 106b. The operative detector 106a is shaped as a disc 402 or other configuration suitable to house sensor(s) (e.g., one or more sensor devices, as hereafter further detailed). The disc 402 of the wand 106 can have three-dimensional shape, such as in a semi-conical or other 3-dimensional shaped housing, in order to accommodate sensors for multiple directional dimensions (as hereafter detailed). The handle 106b is an extension 404 of the disc 402, formed with or connected to the operative detector 106a. The extension 404 is suitable for manual handheld manipulation of the device 400.

If and as applicable for certain applications or as desirable, the detector device 400, via the handle 106b or otherwise, is connectable to or associable with supporting structures, such as frame, conveyor, or similar automated or manual movement feature. Alternatively, the table 110 (shown in FIG. 1) may be manipulable to move the patient 104 relative to the detector device 400. In any event, the detector device 400 is moved in relation to the patient 104, or vice versa, in a manner to allow the device 400 to detect any extraneous item 102 via passage externally to the patient 104 of the detector device 400 in the vicinity of the item 102 in the patient 104.

Continuing to refer to FIGS. 4A-D, in conjunction with FIGS. 1-3, the extension 404 is manually held by an operator (not shown), such as a medical professional, in use to detect any extraneous item 102 (shown in FIG. 1) inside a patient 104 (shown in FIG. 1). The operator, gripping the extension 404, moves the disc 402 of the wand 106, above and around the body of the patient 104 (shown in FIG. 1). In the vicinity of any extraneous item 102, the sensor(s) of the disc 402 of the wand 106 senses indication of the extraneous item 102. The wand 106, upon sensing of an item 102 during detection operations, signals that the item 102 is sensed. The signaling by the wand 106 is, for example, an audible, visual, electronic or other alert or action, indicative of presence of the sensed item 102 in the vicinity of location of the item 102 in the patient 104.

### Tags and Sensor(s):

The detector device 400, i.e., such as the detector wand 106, includes one or more sensors contained internal to the disc 402 (or other housing or dimensional structure, as applicable). The sensor detects a magnetic field strength (e.g., field strength vectors of 3-dimensions) at a location in space. The item 102 has a magnetic tag that is detectable by the sensor by virtue of magnetic field strength at the location in space of the tag (as hereinafter discussed). Magnetic tags are tags applied to medical sponges or other medical equipment and tools prior to use and entry internally to the patient 104. For example, materials useable as magnetic tags include ferrous or non-ferrous permanent magnets (e.g., any and all types and need not be generative material), soft ferrite materials (e.g., also known as non-permanent magnetic materials) that respond to existing (e.g., magnetic field of the earth) or induced magnetic fields, and active materials that produce a (non-resonant) magnetic field when stimulated. Other tag materials are alternately or additionally employable, such as other magnet materials or magnetically stimulatable materials, or otherwise detectable materials (e.g., which can be detected through sensing of physical property by applicable sensor, without resonation, radiative or electrical emission, or other activity of the tag).

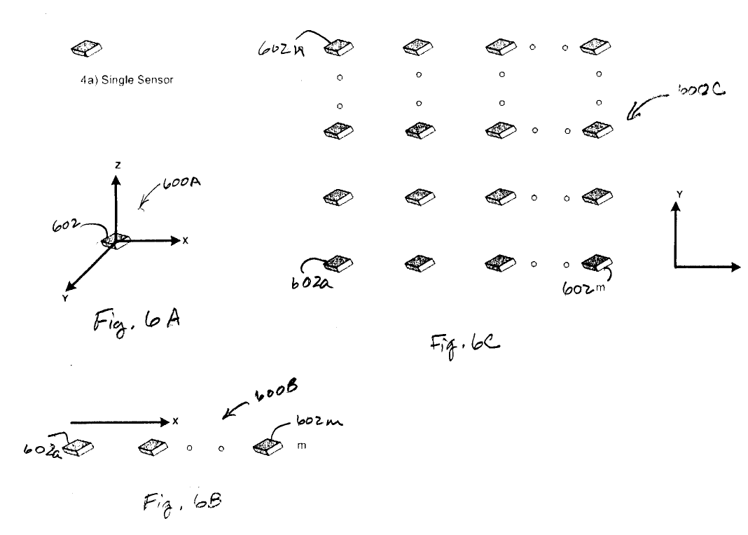

Referring to FIGS. 5A and 5B, in conjunction with FIGS. 1-4, simplified illustrations of a magnetic field 500 exhibited at an operating table 1 10 and patient 104 (patient 104 not shown to simplify Figure) are shown as examples in two situations. In a first situation in FIG. 5A, an ambient magnetic field 502a is formed of a remnant magnetic field of the table 110 and patient 104 in conjunction with the earth's magnetic field. In a second situation in FIG. 5B, the magnetic field 500 includes a magnetic anomaly 502b resulting from a change/affect to the ambient magnetic field 502a of FIG. 5A. In FIGS. 5A-B, the patient 104 is not shown in detail for simplicity of illustration; however, in detection operations, the patient 104 is positioned on the table 110 and, together, the patient 104 and the table 110 with the earth magnetic field, effects the ambient magnetic field 502a.

The sensor(s) of the detection device 400 is a conventional magneto resistive sensor, such as the 1 and 2 axis magneto-resistive sensors available from Honeywell International Inc., Model No. HMC1001/HMC1002. The sensor is, for example, a 3-axis (i.e., x, y and z dimensions) vector sensor. The sensor measures the relative orientation of magnetic field strength vectors (i.e., x, y and z directions) to the axis of the sensor. Magnets, such as the tag for the item 102, generate a magnetic field with north and south pole (i.e., two-pole). Magnetic field strength of these two-pole elements are field line vectors, and can have an unknown orientation to the device 400 (e.g., because of position of the tag of the item 102 in the patient 104). The sensor can detect the magnetic strength vector at each spatial location of the vector, provided the sensor is oriented in suitable manner to the magnet poles of the tag. The 3-axis sensor, capable of sensing strength vector in 3-dimensions, measures the relative orientation of the magnetic field vector at each location of the sensor, by virtue of the 3-dimensional sensing capability. For purposes of description herein, relative orientation at any particular location, of the magnetic field vector to the 3-axes of the sensor, is denoted as Mᵥ for the magnetic field vector.

Movement of the sensor (via movement of the detection device 400 containing the sensor) in relation to the magnetic field 500 of FIGS. 5A or 5B, can yield a measure of the magnetic field vector detected at each spatial location of the sensor. The sensor outputs a voltage proportional to intensity of the magnetic field (i.e., magnetic field strength vector) along each axis of sensitivity of the sensor (i.e., vector intensity in x, y and z-dimensions, if a 3-axis sensor). The voltage output by the sensor at each spatial location over the magnetic field 500 is, for each axis of the sensor, determined, amplified and digitized. The digitized data is delivered to a central processing unit (CPU) of a computer. As the sensor is moved at each spatial location in the magnetic field 500 (e.g., across the body of the patient 104 on the table 110), the respective data for the spatial location is sampled. Continuous sampling of the output of the sensor on each axis (e.g., x, y and z-dimensions) provides information for the CPU to calculate and, thus, measure the relative field strength vectors in time and space. Herein, the array of field strength vectors at each spatial location of the sensor is denoted as Mᵥ(t).

Two or more sensors differently spatially oriented in the device 400 (e.g., having differing position in space in the configuration for the device 400) each detect respective vector values for the respective spatial locations at each instant. A differential or gradient for each spatial location at each instant is derivable from the respective magnetic field strength vectors detected by the sensors. Sensor movement, by scan via the device 400, throughout the magnetic field 500, detects and collects vector data for the respective sensors at each respective spatial location in the magnetic field 500. The array Mᵥ(t, x, y, z) represents a first derivative of magnetic field strength vectors at each instant/location. This first derivative gives a unique magnetic field vector gradient value at each spatial location within the magnetic field 500 at the point in time of sensing.

In operation of the device 400, the ambient magnetic field 502a of FIG. 5A is measured in a scan by the device 400 to determine relative magnetic field strength vectors for each of the two or more sensors. The respective magnetic field gradient values for each location of the ambient magnetic field 502a provide a mapping representation of the magnetic field strengths at each location. This scan gives a base or control determination of the ambient magnetic field 502a for the table 110 and patient 104, together with the earth's magnetic field. The base determination is made, for example, just prior to surgery and introduction of extraneous items 102, such as medical sponges, instruments, and the like.

Subsequently, such as during or at pre-closure (or after closure) of the medical surgery, a next scan of the magnetic field 500 by the device 400 again detects the relative magnetic field strengths at the respective sensors at each instant and spatial location. Respective first derivative gradient values calculated for each spatial location are comparable to those of the base/control for the ambient magnetic field strengths 502a. The magnetic field 500 containing the magnetic anomaly 502b of FIG. 5B, when measured and compared to the base/control measure of the ambient magnetic field 502a, shows a different result on comparison of magnetic field gradient values in the location of the anomaly 502b. If there is not any presence of any tag of the item 102, then the determination is substantially the same as that for the ambient magnetic field 502a, and the conclusion can be made that there is not any item 102 in the magnetic field 500 (for example, no item 102 with tag is left in the body cavity of the patient 104). On the other hand, if the tag of the item 102 is present (e.g., remaining in the patient 104), then the determination shows the different gradient value resulting because of the magnetic anomaly 502b at the spatial location of the item 102 in the magnetic field 500 where the magnetic anomaly 502b differs from the previously detected ambient magnetic field 502a.

If a threshold level difference of gradient value is determined at a spatial location in separate scans by the device 400, the device 400 signals to alert as to presence of the tagged item 102. The tagged item 102 is further locatable by moving the device 400 in the area of the threshold level difference of gradient value, in order to approximate via signals of the device 400 a corresponding location of the tag of the item 102 in the patient 104. Applicable threshold levels depend, in any situation, on various factors, including, for example, size of magnetic tag and the like. Appropriate thresholds are configured for the device 400, in order to appropriately detect extraneous items 102 from the corresponding magnetic field strength gradients.

Referring to FIGS. 6A-C, in conjunction with FIGS. 1-5, a sensor 600A, in FIG. 6A, is a single sensor device 602 that senses and outputs voltage(s) indicative of 3-axes of measurement of magnetic field strength vector (e.g., x, y and z-axes vectors, and measurement for each). A one-dimensional array of sensors 600B in FIG. 6B, such as sensor devices 602a-m that are equi-spaced linearly in a single direction, yield magnetic field gradient values along a single-dimension per the 3-axes of measurement by each of the devices 602a-m (e.g., along direction of movement of the array 600B in the x-direction, and gradient value (x) measurement at each spatial location in the direction of movement). The one-dimensional array 600B provides Mᵥ(t) as a function of F(x) in FIG. 6B.

In FIG. 6C, a two-dimensional array of sensors 600C, such as sensor devices 602a-m,n, are equally spaced in a pattern (e.g., square, m×n) over x-axis by y-axis. Each sensor device 602a-m,n of the two-dimensional array 600C yields magnetic field gradient values along two-dimensions per the 3-axes of measurement by each of the devices 602a-m,n. Gradient value (x, y) measurement is made for each spatial location in the x-y plane during movement of the array 600C over the plane. The two-dimensional array 600C provides Mᵥ(t) as a function of F(x,y).

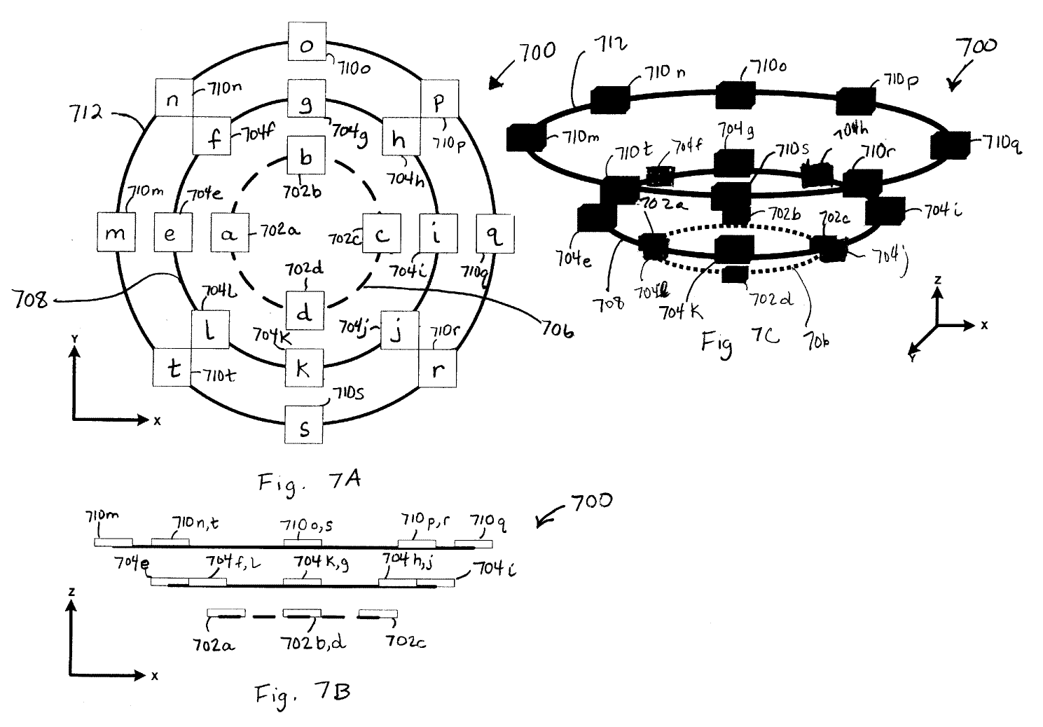

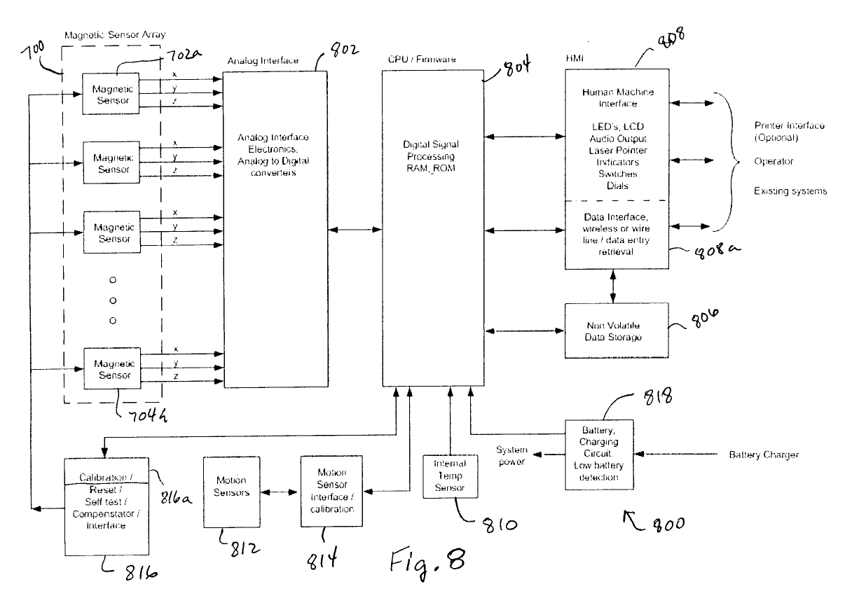

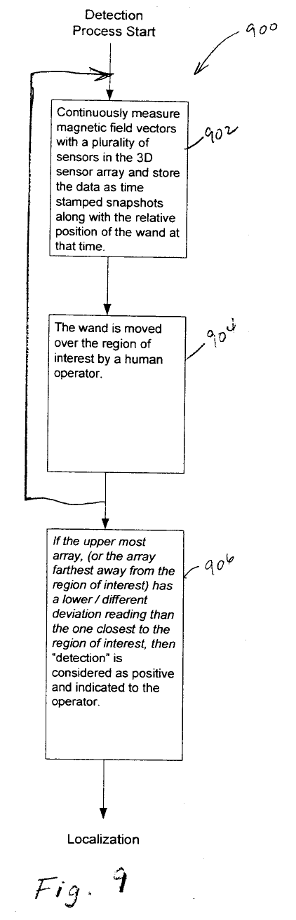

Referring to FIGS. 7A-C, in conjunction with FIGS. 1-5, a three-dimensional array of sensors 700, includes two-dimensional array 702a-d (x-y plane), two-dimensional array 704e-l (x-y plane), and two-dimensional array 710m-t (x-y plane), each array being positioned respectively in a third dimension (z). The array 700, in top downward view in FIG. 7A, includes sensors 702a-d spatially equi-distance arranged around a smaller circular pattern 706, sensors 704e-l spatially equi-distance arranged around a larger circular pattern 708, and sensors 710m-t spatially equi-distance arranged around a largest circular pattern 712. The patterns 706, 708, 712 have common vertex. The 3-dimensional array of sensors 700 is included in the wand detector 106. For example, the disc 402 forming the operative portion 106a of the detector 106 houses the array 700.

In FIG. 7B, each of the x-y planar circular patterns 706, 708, 712 of the sensors 702a-d, sensors 704e-l, sensors 710m-t, respectively, is disposed vertically along different locations of the z-dimension. If the wand detector 106 is held by an operator above a patient 104, the detector 106 is located in relative position (as shown in FIG. 1) such that the two-dimensional array 702a-d, the two-dimensional array 704e-l, and the two-dimensional array 710m-t are in the relative position of FIG. 7B. FIG. 7C illustrates each of three-dimensions of the array 700. The wand detector 106 includes the array 700. The three-dimensional array 700 provides Mᵥ(t) as a function of F(x,y,z).

In the array 700, the sensors 704e-l, 710m-t of the circular patterns 708, 712, respectively, are considered to detect boundary conditions of magnetic field vectors around the sensors 702a-d of the circular pattern 704. The bounded area is approximated to be a generally cylindrical volume extending in z-dimension (in the Figures) below the array 700 assembly and encompasses an area of interest for detection. In effect, when boundary conditions around an enclosed volume are known, the magnetic field gradient (i.e., the effect of magnetic fields) coming from outside of the bounded volume are calculable. Thus, the outside boundary conditions, by comparison to inside measured values, yields a difference value that cancels out ambient magnetic field vectors (i.e., effects) as measured and calculated at the boundary. Therefore, readings of the sensors 702a-d during scan process with the array 700 represent magnetic field value (i.e., effect) generated inside the approximated cylindrical volume for the array 700 (i.e., with cancelled “ambient”). The representative field value is comparable, through subtraction calculation, to determine gradient value for inner sensors 702a-d, in effect, with cancelled ambient magnetic field vectors as measured and calculated at the boundary of the bounded volume. Thus, inner sensor 702a-d readings are comparable in scan in presence of an anomaly, to be in excess of a threshold value for indicating presence of the anomaly in the bounded volume space for the patient cavity. The excess of threshold, so indicated at any point of scan, indicates that an item is then-present in approximately the central vicinity of the cylindrical volume extending in the patient (i.e., in imaginary space for the volume in z-axis extension into the patient).

In the array 700, the sensors 702a-d of the smaller circular pattern 706, the sensors 704e-l of the larger circular pattern 708, and the sensors 710m-t of the largest circular pattern 712, respectively, can have different threshold level setting in the device 400. This can reduce false positive detection of any anomalies 502b without compromising detection of relevant anomalies 502b that are due to presence of tagged item 102.

Alternately, other configurations and arrangements of arrays of sensors are possible, as those skilled in the art will know and appreciate. Additional or fewer sensor devices are possible. Additional, fewer or otherwise patterned or configured arrays are possible. For example, linear array, or even single sensor, may be incorporated with multiple other arrays or arrangements. Each different sensor, and different array of sensors, will have unique characteristics and, thus, will provide particular detection effects and capabilities. Appropriate sensing and computational details are unique to each respective design and configuration. Moreover, the various alternatives and options in sensor(s), arrays, and relative arrays and sensor(s) can be selected for particular application, needs, economics, effectiveness, and other considerations. The array 700 of FIGS. 700A-B in the wand detector 106 is, nonetheless, suitable and capable for operations as desired for the purposes hereof, as used and described herein.

### Detection System:

Referring to FIG. 8, in conjunction with FIGS. 1-7, a detection system 800 includes the wand detector 106. The wand detector 106 includes a housing, such as in FIGS. 1-4 having an operative portion 106a, for example, the disc 402, and a handle 106b, for example, the extension 404. The wand detector 106 includes in the operative portion 106a the array 700 of sensors of FIG. 7. As just described, this array 700 is 3-dimensional in orientation of respective sensor devices 702a-704h and each such sensor itself is capable of magnetic field strength vector detection in 3-dimensions (x, y, and z) at each spatial location of the sensor.

In the detection system 800, each sensor device 702a-704h of the array 700 is communicatively connected to an analog interface 802. Output voltage signals of each respective sensor device 702a-704h, indicative of values of magnetic field strength vector in 3-axes of the sensor (i.e., x, y, and z) at each then-present spatial location of the sensor, are continuously streamed to the analog interface 802. The analog interface 802 includes analog-to-digital converters (not shown in detail) for the streamed signals.

The analog interface 802 is communicatively connected to a digital signal processor 804. The digital signal processor 804 includes a CPU, memory (e.g., RAM, ROM), and software for control. The digital signal processor 804 is communicatively connected to a non-volatile memory 806 for data storage. Software is stored in the memory 806 to control the CPU for applicable data collection, calculations, data storage and the like. Software control is hereinafter further described as relative to detection operations. Of course, a wide variety of options, alternatives and additions are possible for the software, as those skilled in the art will understand, and all are included herein. Further, circuits and other communicative and logical devices and schemes can add to, aid in, substitute for, or otherwise provide or alter the operations as desired in application, as will be understood, and all such variations are likewise intended as included here.

The digital signal processor 804 is communicatively connected to a human-machine interface 808, for example, including input and output devices, such as audio outputs, audio indicators volume, on/off switch, visible outputs, LEDs, LCD or other lamps or light indicators, calibration indicator, low battery indicator, LCD character display indication, laser pointer (e.g., located in center of disc 402 of the wand detector 106 active to indicate approximate location at the patient 104 of any item 102 detected), other indicators, data entry interface and devices (e.g., interface to computers, keyboards, LAN and/or other standard interfaces), switches, dials, keys, keypad, and/or the like. Additionally, the human-machine interface 808 includes, for example, printer and other reporting devices and connects to ancillary peripherals or other devices, networks, or the like, including, such as, barcode reader for patient data, encrypted data retention, external non-volatile memory, self tester and capability, data encryptor, time stamp, and other manual or automated features, such as rechargeable battery, automatic calibrator and calibration notification. The processor 804 communicates with and through, and the software controls and is controlled by, at least certain of the inputs and outputs via the human-machine interface 808, as well as other possible peripherals and the like. For example, the human-machine interface 808 includes a data interface 808a for wired, wireless or other data input/output and retrieval in the system 800.

A battery 818 of the system 800 is connected to the processor 804 and other powered elements of the system 800 (e.g., lights, sound devices, sensors, etc.). The battery 818 powers the system 800 for scan operations. The battery 818 includes, for example, charging circuitry and connections, low battery detection and indicator, and the like.

System Check/Set:

A system check/set device 816 communicatively connects to each respective sensor device 702a-704h of the array 700, and to the processor 804. The device 816 checks the system 800, either manually, automatically or combinations, to set up the system according to calibration criteria, via processing by the processor 804 and software therefor, and according to sensor device 702a-704h and array 700 conditions and applicable use. In effect, the device 816 checks the system 800 during scan usage, to adapt for conditions and correction to ambient determinations.

Temperature Sensor:

The detection system 800 also includes a temperature sensor 810 communicatively connected to the processor 804. The temperature sensor 810 is, for example, incorporated within the wand detector 106 at the array 700. High resolution sensor devices typically have sensitivity to temperature variations. Calibration data for the sensor devices, and the system 800 as a whole, can vary according to temperature variation. The temperature sensor 810 signals the processor 804, for example, a digital signal indicative of temperature value at each spatial location of the wand detector 106. Temperature data is stored in the non-volatile memory 806 of the system 800. The data is employed by the processor 804 and software to adjust signal processing, for example, of signals from the sensor devices, based on actual temperature of the detector 106 during use.

Motion Sensor:

A motion sensor 812 is communicatively connected to the processor 804, such as via a motion sensor interface 814. The motion sensor 812 is, for example, an accelerometer. The motion sensor 812 detects information in respect of the actual motion of the wand detector 106 during scanning for detection. The actual motion information so detected by the motion sensor 812 is digitally signaled to the processor 804 via the motion sensor interface 814. Procedural parameters for appropriate or desired motion of the detector 106 in scanning procedures, such as to ensure applicable and verified results of operations, are input to and saved for operations of the software and processor 804 of the system 800. The actual motion information is compared to the procedural parameters by the system 800, and allows the system 800 to discern faulty or failure motions and results.

In particular, scans with the detector 106 must be properly made or otherwise the system 800 indicates “Scan Fail”, such as by audible or visible signal from the detector 106. Typical parameters that are verifiable for each scan pass by the detector 106 include, for example, scan speed, scan attitude (e.g., angle at which the detector is held/moved in the scan), scan coverage (e.g., indicative whether entire area was properly covered in scan), actual motion of the detector 106, scan time elapsed (e.g., amount of time spent in the scan), and others. Software and processor 804 operations are accordingly affected, as per the particular embodiment and application.

Calibration Fixture:

A calibration fixture 816a of the system 800 is communicatively connected to each respective sensor device 702a-704h of the array 700, and to the processor 804. The calibration fixture 810a includes, for example, a known magnetic strength source. The calibration fixture 810a signals the processor 804 to initially set operations, per software and processor 804 configuration and based on the known magnetic strength source. The calibration fixture 810a is reliably useable only if there are not any significantly fluctuating ambient or strong ambient forces present.

### System Operations:

Referring to FIG. 9, in conjunction with FIGS. 1-8, in operation, the system 800 performs a method 900 of detecting. The method 900 commences in a step 902 of moving the detector 106 of the system 800 over an area of a magnetic field 500 of ambient magnetic field strengths 502a. The step 902 continuously measures magnetic field vectors, at each spatial location, for the magnetic field 500 of ambient magnetic field strengths 502a.

In a step 904, the detector 106 is moved (in space) over an area/region of interest of the patient 104. The step 904 is performed by an operator manually handling the detector 106. Data of the measurement in respect of each spatial location for the movement of the detector 106 is stored in memory as time-stamped snapshots of relative position of the detector 106 of the system 800 in the magnetic field 500. The measurements of magnetic field vectors at each spatial location are taken synchronously by the sensor devices in the step 902. The data set of time-stamped snapshots for the scan in the step 902, i.e., at each time and spatial location, provides a baseline measurement for the method 900. Processing by the system 800 of the baseline data set yields a 3-dimensional mapping of the ambient magnetic field gradient profile.

Thereafter, the step 902 is again performed at a different time (e.g., prior to closure on completion of surgical procedures) to detect presence of any remaining extraneous item 102 within the magnetic field 500. As previously described, the magnetic tag of the extraneous item 102 is detected as a magnetic anomaly 502b of the magnetic field 500, in comparison to the ambient magnetic field strengths 502a for the magnetic field 500 previously determined as the baseline. The magnetic tagged item 102 effects a gradient determination at the location of the item 102 that, compared to the baseline gradient for the location, exceeds an applicable threshold level of the device 400.

As the device 400 is moved in the step 904 over the region of the item 102, the device 400 signals when the threshold level is triggered because of the different gradient determination in the location. The corresponding location of the item 102 in the vicinity of the signal by the device 400 is further positioned in the patient 104 by moving the device 400 to various positions and noting signaling. Through the signaling by the device 400 in several positions with respect to the patient 104, the item 102 (i.e., tag of the item 102) is pinpointed in approximate position and location of the patient 104. The item 102 is then readily retrieved.

A step 906 is performed by the device 400 having the different threshold level settings for the sensor devices 702a-d and 704e-h of the respective circular patterns 706 and 708. The step 906 by the device 400 checks for false positive results if the threshold level is exceeded in the scan. In particular, in order for the device 400 signal that an item 102 is detected, the device 400 considers whether the respective sensor devices 702a-d and 704e-h indicate a differential value therebetween that exceeds a set value for the device 400. For example, if the differential value does not exceed the set value, then there is not any positive detection. On the other hand, if the differential value exceeds the set value, then a positive detection is signaled by the device if the threshold level is then exceeded in the steps 904, 906.

### Probe Detector:

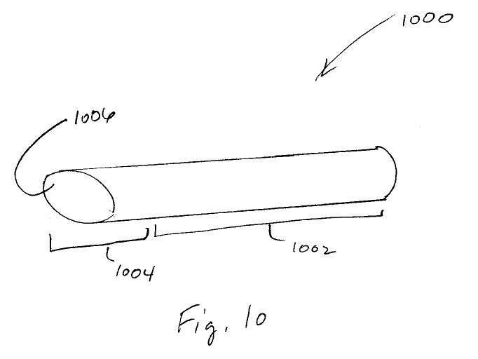

Referring to FIG. 10, a probe detector device 1000 includes either a single sensor or a two-dimensional array of sensors (not shown in detail). FIGS. 1-3 illustrate the probe detector device 1000 in use as detector probe 108 in the Figures. The probe detector device 1000 in such use is highly manipulable and can extend into regions within the patient 104 in order to more specifically find any extraneous item 102 by movement of the device 1000 into close proximity with the item 102. For example, the device 1000 can be employed if an item 102 is indicated by the wand detector 106 as present, but requiring further and more pointed search to locate.

The device 1000 is formed as a cylinder-shape. The device 1000 has a handle portion 1002 and an operative portion 1004. The handle portion 1002 is manually grippable for scanning movement, to selectively manipulate the operative portion 1004 during scan, including within the body cavity of the patient 104. The operative portion 1004 includes the sensor(s). The device 1000 at the operative portion 1004 is substantially rounded with a point-like end 1006. The end 1006 allows precise probe scanning into, through and between internal bodily features of the patient 104. The rounded edges of the end 1006 limit concern of cutting, puncture, or other bodily damage in use. Multiple ones of the device 1000, having different size and sensor counts, provide precision detections in virtually any location, notwithstanding adjacent bodily features of the patient 104. For example, the device 1000 can be formed as quite small in cylindrical size and of length as desired for probing to detect in minute spaces and spots. Of course, in more spacious bodily areas, the device 1000 can be larger or otherwise differently formed. The device 1000 can have shape, flexibility and the like, as needs and application require.

If a single sensor is included in the device 1000, such as in a small shaped form for the cylinder operative portion 1004, the device 1000 determines an ambient read of the scalar value of the magnetic field strength in the vicinity of the area being investigated. The area is scanned by physically probing/moving the operative portion 1004 in a controlled manner to measure respective magnetic field strength at respective spatial locations. Alternately, the single sensor of the device 1000 is calibrated prior to scanning, such as with respect to a value relative to a known magnetic strength source or the like. If the device 1000 is so pre-calibrated, then the pre-scan for calibration required in other embodiments is not needed. Instead, the device 1000 is set (or settable) based on this reference source via an established threshold value setting related to a difference in magnetic field strength that would be expected in presence of a magnetic tag of a sponge or implement. For example, if the threshold value for the setting is reached or exceeded (or within a tolerance range therefor), then the device 1000 signals to indicate apparent presence of the tagged item. The threshold value setting can be set by manufacture, for example, based on “typical” environment, or the value setting can be made by calibration at location at the time of testing, or other implementations are possible.

In subsequent scanning (such as to detect pre-closure or the like), the scalar value measured at a spatial location in the vicinity of interest as the ambient read, is compared with then-detected scalar value of the magnetic field intensity at the spatial location. If the scalar values differ significantly, in excess of threshold levels for the device 1000, the device signals an alarm. The alarm indicates detection of the item 102. The threshold level of the deviation of the respective scalar values (i.e., ambient and the then-detected value) is adjustable for desired sensitivity of detection.

A single dimensional array of sensors, such as the array 600B of FIG. 6B, can be included in the device 1000 in certain embodiments. Such an array will, for example, allow false positive clarity because of varied spatial positions of the sensors and/or further detection capability through multiple sensors. Detection is confined to single dimension, however, in accord with particular directional orientation at each instant of the array of the operative portion 1004 and the orientation of the item for detection.

A two-dimensional array of sensors, such as the array 600C of FIG. 6C, can be included in certain other embodiments of the device 1000. For example the array 600C can include two sensors, one as a reference and the other as a measurement device. An absolute value of the difference in scalar values detected by the respective sensors at each spatial location indicates magnetic field gradient at the location. If this absolute value of the difference in scalar values differs significantly from the ambient scalar value of magnetic field intensity, the device 1000 signals an alarm to indicate detection of the item 102. The particular deviation in scalar values that is the threshold is adjustable to vary the detection sensitivity as appropriate.

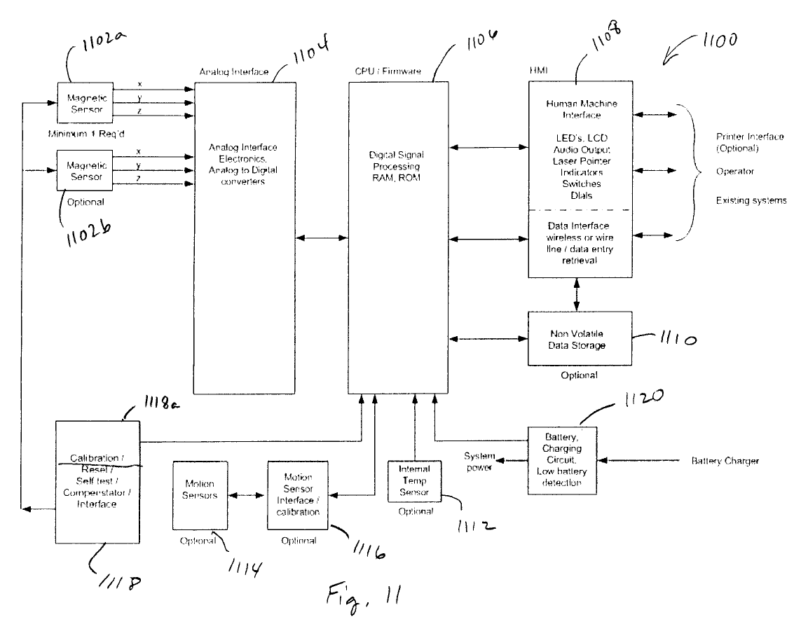

Referring to FIG. 11, the device 1100 includes similar features to the device 800 of FIG. 8. The single sensor 1102a, or one-dimensional array of sensors 1102a-b, as applicable, are communicatively connected to an analog interface 1104 that includes A/D converters for receiving and digitizing voltage output signals of the sensors 1102a,b. The voltage output signals, as previously described, are indicative of magnetic field strength vectors in 3-dimensions (x,y,z) at each spatial location of each sensor 1102a or b.

A digital signal processor 1106 is communicatively connected to the analog interface 1104. The processor 1106 communicatively connects to peripherals, input-output devices and the like, such as the human-machine interface 1108 previously discussed. A non-volatile data storage 1110 is communicatively connected to the processor 1106 and the human-machine interface 1108.

The device 1100 also can include a temperature sensor 1112, a motion sensor 1114 and motion sensor interface 1116, and system check/set device 1118, calibration fixture 1118a and the like. Each of these elements 1112, 1114, 1116, 1118, 1118a is communicatively connected to the processor 1106, and the check/set device 1118 and the calibration fixture 1118a are each also communicatively connected to the sensors 1102a,b, as applicable. A battery 1120, and appurtenant features, is connected to and powers the device 1100 and its various powered elements.

### Detector and Signal Veracity:

In the foregoing systems and methods, signal veracity of magnetic intensity is important to proper detection of the item 102. To increase sensitivity of the particular embodiment of the system in an applicable design, any plurality of sensors and sensor arrays are matched in physical orientation and magnetic responsivity on each dimensional axis. Higher system accuracy and calibration effectiveness is achieved, and ambient magnetic field vectors are better discounted/cancelled in subsequent determinations by such balanced/matched embodiment. False positive incidence is, thus, reduced without compromise of detectability.

In the wand detector 400 of FIGS. 4A-D, the three-dimensional arrangement of sensors measures and allows processing of magnetic field gradients in either Cartesian coordinate system (x, y, z) or in polar coordinate system (R,φ,⊙) with same results. In the arrangement, controls adjust sensitivity, by varying threshold limits for magnetic field gradient in the device 400. Higher sensitivities (lower threshold) allow greater range for detection in distance of the device to the item. Lower sensitivities (higher threshold) reduce the range of detection.

### Tags:

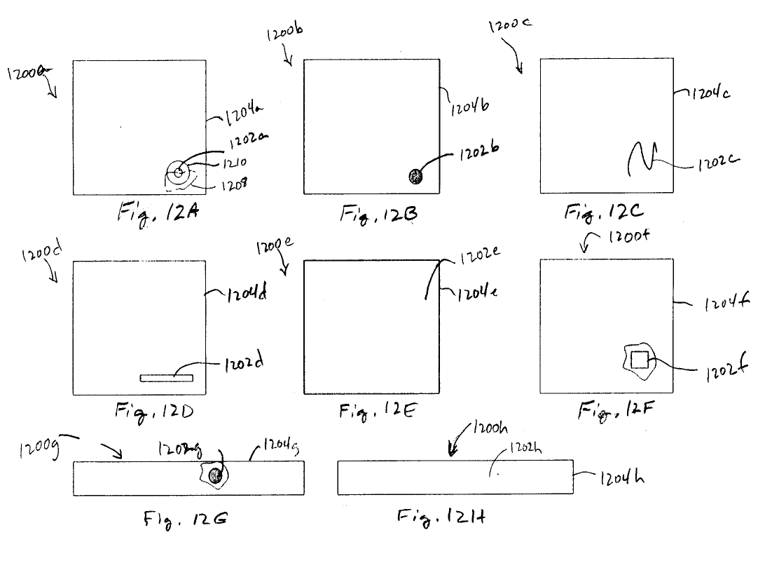

Referring to FIGS. 12A-H, various designs, configurations and types of tag 1202a-h are attachable to items 1200a-h, such as the extraneous items 102 of prior Figures and description. For example, medical/surgical sponges 1204a-f and instruments 1206g-h are included herein as these types of extraneous items that are introduction from external the body of the patient 104 and into the body of the patient 104, and/or otherwise used in the operative vicinity during medical surgery and subject to potential misplacement. As is conventional, numerous sponges, instruments, devices and the like can be used in medical/surgical procedures. These sponges and instruments must be accounted for, in order to ensure retrieval from within the patient 104 prior to surgical completion with incision closure. Further, there are other situations and instances in which a tag must be tracked within a human or animal body. Magnetic tags, such as the tags 1202a-h of FIGS. 12A-H, are locationally detected by the foregoing detection devices and/or other comparable magnetic field affective detection systems and methods.

Referring to FIG. 12A, an item 1200a includes a spherical, bead-shaped magnet tag 1202a sewn to a sponge 1204a. Threads 1208 bind the tag 1202a to the sponge 1204a, such as in a corner area of the sponge 1204a surface. In certain embodiments, a sewn-in pocket or cover patch 1210 of sponge material is sewn to enclose and retain the tag 1202a affixed to the sponge 1204a. The tag 1202a is alternately formed as a circular disk shape magnet in the embodiments.

Referring to FIG. 12B, another item 1200b includes a sponge 1204b and an adhesively attached magnet tag 1202b, such as the tags of the Figures. A medical grade adhesive fixes the tag 1202b to a location, such as a corner, of the sponge 1204b.

Referring to FIG. 12C, an item 1200c includes a different magnetic tag 1202c formed of a wire magnet. The wire-shaped and formed tag 1202c is sewn into a sponge 1204c.

Referring to FIG. 12D, another item 1200d is a sponge 1204d that includes a magnetic tag 1202d. The magnetic tag 1202d is a strip of magnetic material that is printed or attached onto the sponge 1202d. Heat or pressure applied to the magnetic material at the sponge 1204d fixes the tag 1202d to the sponge 1204d. The tag 1202d is, for example, rubber or plastic magnet material on application of heat, pressure or the like, and interstitially invades weaved threads or other material of the sponge 1204d. The tag 1202d thereby attaches to the sponge 1204d.

Referring to FIG. 12E, an item 1200e is an alternative design of a sponge 1204e that is formed with magnetic material 1202e deposited onto or integral in the sponge material. For example, the sponge 1204e is a weave of quite flexible wire, rubber or plastic threads having magnetic effect. Alternately, a sponge material is coated or covered by the magnetic material 1202e. In the sponge 1204e, the sponge 1204e is, itself, the detectable tag because of the magnetic material 1202e thereof.

Referring to FIG. 12F, another item 1202f is a sponge 1204f and affixed magnetic tag 1202f. The magnetic tag 1202f is formed of an active non-resonant magnet. The tag 1202f is attached to the sponge 1204f, for example, by medical grade adhesive, sewn to the sponge material or within a pocket or cover sewn to the sponge material, or otherwise fixed to the sponge 1204f in manner like or similar to those of the other Figures.

Referring to FIGS. 12G-H, items 1200g-h are medical instruments 1202g-h that include magnetic tags 1202g-h formed to the instrument 1204g. In FIG. 12G, the instrument 1204g is, for example, scissors, clamp, poker, or other as may be used in a surgical procedure and introduced near or into the patient 104. A magnetic tag 1202g of the item 1200g is adhered to the instrument 1204g. For example, the tag 1202g is fixed to an outer surface of the instrument 1202g by medical grade adhesive or the like.

In FIG. 12H, the item 1200h is also an instrument 1204h used in a medical or surgical procedure. The instrument 1204h is made of, or includes in the instrument 1204h structure, a magnetized or ferrous steel, such as stainless steel, or other magnetic material. The instrument 1204h, itself, serves as a magnetic tag 1202h by reason of the magnetic effect of the material of the structure.

All of the foregoing magnetic tags, and items (such as sponges and instruments), are examples of possibilities for detectable tagged extraneous items. Other arrangements, configurations, materials, adherents, fixtures, and the like will be known and understand, and all are included herein. The magnetic tag of the item, is in any event, detectable because of the magnetic anomaly caused thereby in the magnetic field. The detectors, previously described, find and locate the presence of the magnetic tag (and, consequently, the item having the tag) as per this description.

In certain embodiments of the systems and methods for detectors, detections, and tags, distinction (i.e., differentiation) between different tags can be made or ascertained between and among pluralities of the tags, by applicable respective tag size and shape. The size and shape effects the magnetic anomaly created in the magnetic field by the presence of the tag (and, consequently, by the presence of the relevant item that has the tag). In particular, any larger/smaller or peculiarly-shaped tag effects different characteristics to the magnetic anomaly, from the characteristics of magnetic anomaly effected by another separate smaller/larger or differently shaped tag. Also, magnetic field strengths of the magnet materials of respective tags can differ, thereby yielding distinct and differentiable magnetic anomaly effects.

This differentiating anomaly effect of respective tags is employable in the systems and methods to provide knowledge of which tag (and, consequently, which item) is detected in any detection incident. If particular tagged items are used in surgical procedures in a certain area/region of the patient 104, other particular tagged items used in a separate area/region in the procedures can be distinguished. Moreover, in quite sensitive detections by the systems and methods, discernment between each respective tag (and, consequently, each respective item) can be made.

Each different tag effects a unique magnetic characteristic. The unique magnetic characteristic is detectable by the systems and methods as a unique and distinct magnetic anomaly. Thus, each tag causes a unique magnetic anomaly. The particular magnetic anomaly for a given one of the tags is sometimes referred to as the “Magnetic Signature” of the tag. The Magnetic Signature of each tag is unique and distinct from the Magnetic Signature of other tags. This Magnetic Signature is detectable by an array of two or more sensors. In systems and methods operated with adequate sensitivities to detect the unique and distinct respective Magnetic Signatures, discernment and determination of which tag, and which item having the tag, is possible. Details of tag and item identification, by respective Magnetic Signature, allow for a wide variety of options, alternatives, additions, modifications, and other possibilities as will be understood, and all of these possibilities and systems and methods therefor are included.

Also, particular and specific location of magnetically tagged items is possible because of the respective Magnetic Signatures of the tags. With a three dimensional array of sensors, any deviation of magnetic field gradient at a spatial location, as compared to baseline ambient map of magnetic field gradient for the location, is detected by one or more sensors of the array. As previously described, magnetic field gradient values for each spatial location in a detection scan are processed from field strength vector measurement at each location. Ambient baseline gradient values for locations are mappingly compared to gradient values for the locations as determined in subsequent scan. The respective ambient gradient values and the present gradient values are each exhibited at each location, and calculated by processing of strength and direction measurements of magnetic field strength vectors, at separate points in time of the respective scans. The detectors of the systems and methods provide visual and audio feedback to the scan operator to indicate spatially where (on which sensor(s)) any deviating anomaly is detected. The anomaly is, thus, detected by vectorally calculating the deviation from ambient.

The three dimensional array of sensors is moveable by the operator in spatial direction indicated by the feedback to the scan operator on deviating anomaly detection. As the array is moved towards and around the anomaly location, feedback of the detector is noted until the array is centered over the anomaly. As centered, the sensors of the array indicate that respective sensors closest to the area of interest for the deviating anomaly exhibit an equivalent deviation of gradient value from ambient. The centered array over the anomaly indicates a close approximation of spatial location of the tagged item, at the center of the array. Approximate depth of the tagged item is calculated by the detector of the systems and methods, from sensor readings of the measured deviation. For example, the calculations of approximate depth use fundamental mathematical equations for electricity and magnetism, as processed by the detector device.

As previously mentioned, on approximate location and depth approximation for a tagged item by the three-dimensional sensor array type of detector device, the probe detector device having a single or two-dimensional capability can be used to further pinpoint spatial location of the tagged item. Thus, the various detectors of the systems and methods are usable in conjunction, or otherwise, to accurately locate tagged items for retrieval. The combinational use of detectors in any circumstance, such as to, first, locate the tagged item with three-dimensional sensor array wand device and, second, more specifically locate the tagged item with one or two-dimensional sensor array probe device, is particularly desirable if tagged items may be visually difficult to find. Such may be the case, for example, in the presence of bodily fluids, behind or between internal organs and features, and otherwise lodged or positioned in the patient 104.

### Magnetic Tagged Item Counter:

Referring to FIG. 13, certain embodiments of detector systems and methods are operable in a counter 1300 that is external to the patient 104 and associated with item 102 disposal or the like after removal from the patient 104. Of course, the counter 1300 can include manual count of items 102, as has been conventionally undertaken. Alternatively or additionally, the counter 1300 is a device 1302, of unitized or of conjunctively operable components. The device 1302 is, for example, incorporated with conventional or other disposal facilities 1304 for sponges, instruments, and other items after use.

The device 1302 includes magnetic sensors 1306 located adjacent an entrance to the disposal facilities 1304, such as at an opening of a disposal chute. A similar device 1302 is additionally locatable at a retrieval chute or bin, and coordinated counting of items retrieved and later disposed is possible in such arrangement at retrieval and disposal locations/chutes. The magnetic sensors 1306 include any of the types as have been previously described, including single sensors or arrays of sensors in any plurality of dimensions as desired for the application.

The magnetic sensors 1306 communicatively connect to an electrical interface 1308. The electrical interface 1308, as has been previously described for similar detection operations and devices, includes analog-to-digital converters. A digital signal processor 1310 is communicatively connected to the electrical interface 1308. The processor 1310 operates to process detected magnetic field effects sensed by the sensors 1306 and indicated to the electrical interface 1308 as corresponding voltage outputs of the sensors 1306. A human-machine interface 1312, including internal and external components and peripherals, input-output, memory and data storage, and the like, communicatively connects to the processor 13 10.

In any instant in time, presence of magnetic anomaly is introduced by any tagged item in range of detection sensitivity of the sensors 1306, the device 1302 registers a count of the tagged item and cumulates the count of all such tagged items during use. In this manner, an automated, accurate count is automatically maintained, such as for tagged items employed in a surgery or the like. Variations, including specific identity of unique Magnetic Signature of tags of items and the like, as well as various detection schemes and steps, all as previously described, are implementable as desired or required.

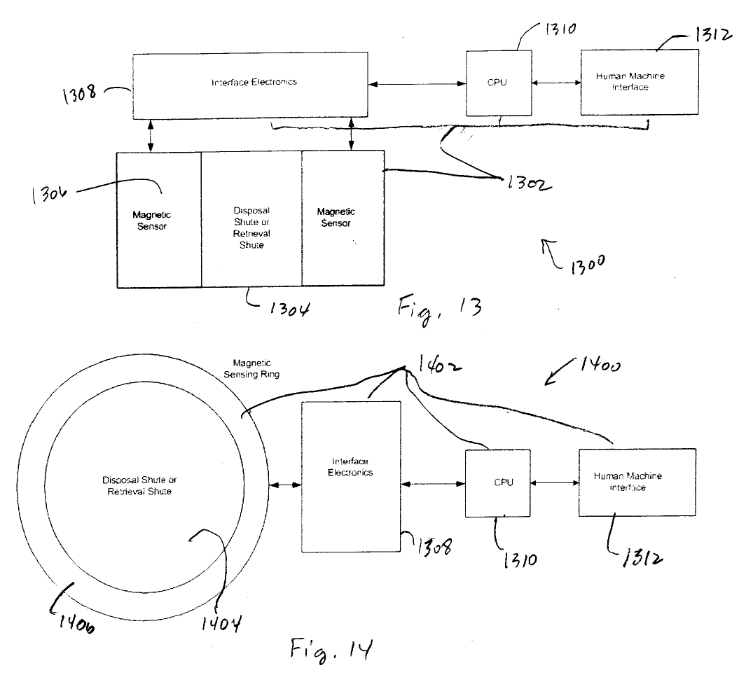

Referring to FIG. 14, an alternative embodiment of a counter 1400 includes a device 1402 comprising the interface electronics 1308, the processor 1310 and the human-machine interface 1312 (the elements 1308, 1310 and 1312 are the same as, of substantially similar to, those of the preceding description with respect to FIG. 13). The device 1402 further includes an alternative sensor 1406 located adjacent an entrance opening of a disposal chute 1404. The sensor 1406 is configured of a single sensor shaped as a ring of the opening of the chute 1404 or, alternately, as an array of sensors located in a circular pattern to form an array ring of the opening of the chute 1404. As tagged items are passed through the sensor 1406, or array as applicable, magnetic sensing of anomaly effect is registered by the device 1402 as count of the respective items. Additional elements, including another same or similar device 1402 associated to a retrieval chute or area, are includable to provide retrieved and disposed counts for items employed in surgery or the like.

In the foregoing specification, the invention has been described with reference to specific embodiments. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of the present invention.

Benefits, other advantages, and solutions to problems have been described above with regard to specific embodiments. However, the benefits, advantages, solutions to problems and any element(s) that may cause any benefit, advantage, or solution to occur or become more pronounced are not to be construed as a critical, required, or essential feature or element of any or all the claims. As used herein, the terms “comprises, “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A magnet detector for locating magnetically tagged items in a surgical patient, comprising:
a three-dimensional (x,y,z) sensor, moveable throughout a magnetic field at the patient, a first output of the sensor represents a first-sensed magnetic field strength vectors for three dimensions at a first spatial location of the sensor and a second output of the sensor represents a second-sensed magnetic field strength vectors for three dimensions at a second spatial location of the sensor;
a processor communicatively connected to the sensor, for receiving the first output and the second output and calculating a value from the first output and the second output representative, respectively, of an ambient scalar (Mᵥ(x)_{ambient}) of the first-sensed magnetic field strength vectors and the second-sensed magnetic field strength vectors;
a storage for the value;
a magnetic field anomaly subsequently induced to the magnetic field at the first spatial location and the second spatial location, the sensor, thereafter moveable throughout the magnetic field of the patient, a third output of the sensor represents a third-sensed magnetic field strength vectors and a fourth-sensed magnetic field strength vectors for three dimensions at the first spatial location and the second spatial location, respectively, of the sensor;
wherein the processor receives the third output and the fourth output of the sensor, calculates a different value from the third output and the fourth output, the different value representative, respectively, of a different anomalous scalar (Mᵥ(x)_{anomaly}) of the third-sensed magnetic field strength vectors and the fourth-sensed magnetic field strength vectors.

2. The magnet detector of claim 1, wherein the processor compares the ambient scalar and the different anomalous scalar, determines a deviation (Δ=|(Mᵥ(x)_{ambient})−(Mᵥ(x)_{anomaly})|) exceeding a threshold for the processor, and signals an output representative of the deviation as indicative of presence of the magnetic field anomaly at approximately the first spatial location and the second spatial location.

3. The magnet detector of claim 2, further comprising:
an alarm connected to the processor, the output of the processor representative of the deviation sounds the alarm indicating the magnetic field anomaly at approximately the first spatial location and the second spatial location.

4. The magnet detector of claim 2, further comprising:
a plurality of the three-dimensional (x,y,z) sensor maintained in an array, each of the plurality communicatively connected to the processor, a respective first output of each of the plurality and a respective second output of each of the plurality, the respective first output and respective second output representative of respective first-sensed magnetic field strength vectors at the first spatial location and second-sensed magnetic field strength vectors at the second spatial location for each respective sensor of the plurality;
the processor receives each of the respective first output and the respective second output and calculates an ambient array value representative, respectively, of an ambient magnetic field strength array (Mᵥ(x,y)_{ambient}) at about the first spatial location and the second spatial location for each sensor of the plurality;
the ambient magnetic field strength vector for each sensor of the plurality is saved in the storage;
the magnetic field anomaly subsequently induced to the magnetic field at the first spatial location and the second spatial location, the plurality of the sensor thereafter moved throughout the magnetic field of the patient, a representative third output of each sensor of the plurality represents a third-sensed magnetic field strength vector and a fourth-sensed magnetic field strength vector for three dimensions at the first spatial location and the second spatial location, respectively, of the each sensor of the plurality;
wherein the processor receives the third output and the fourth output of each sensor of the plurality, calculates a difference array value from the third output and the fourth output, the difference array value representative, respectively, of a different anomalous magnetic field strength array (Mᵥ(x,y)_{anomaly}) of the third-sensed magnetic field strength vector and the fourth-sensed magnetic field strength vector for each sensor of the plurality.

5. The magnet detector of claim 4, wherein the processor compares the ambient array value and the difference array value, determines a deviation (Δ=|(Mᵥ(x,y)_{ambient})−(Mᵥ(x,y)_{anomaly})|) exceeding a threshold for the processor, and signals an output representative of the deviation as indicative of presence of the magnetic field anomaly at approximately the first spatial location and the second spatial location

6. The magnet detector of claim 5, further comprising:
an alarm connected to the processor, the output of the processor representative of the deviation sounds the alarm indicating the magnetic field anomaly at approximately the first spatial location and the second spatial location.

7. The magnet detector of claim 4, further comprising:
a plurality of the array, each array of the plurality respectively positioned as to a third perpendicular dimension (z) to the two dimensions (x,y);
each of the array of the plurality moveable throughout the magnetic field of the patient, a first respective output of each respective array of the plurality represents a first-sensed magnetic field strength gradient for three dimensions at the first respective spatial location of each respective array of the plurality and a second respective output of each respective array of the plurality represents a second-sensed magnetic field strength gradient for three dimensions at the second respective spatial location of each respective array of the plurality;
the processor receives each of the first respective output and each of the the second respective output and calculates an ambient field gradient array value representative, respectively, of an ambient magnetic field strength gradient (Mᵥ(x,y,z)_{ambient}) at about the first spatial location and the second spatial location for each array of the plurality;
the ambient field gradient array value each array of the plurality, and each of the plurality of sensors of each array, is saved in the storage;
the magnetic field anomaly subsequently induced to the magnetic field at the first spatial location and the second spatial location, the plurality of the array thereafter moved throughout the magnetic field of the patient, a third representative output of each array of the plurality represents a third-sensed magnetic field strength gradient and a fourth-sensed magnetic field strength gradient for three dimensions at the first spatial location and the second spatial location, respectively, of the each array of the plurality;
wherein the processor receives the third output and the fourth output of each array of the plurality, calculates a difference gradient array value from the third output and the fourth output, the difference gradient array value representative, respectively, of a different anomalous magnetic field strength gradient array (Mᵥ(x,y,z)_{anomaly}) of the third-sensed magnetic field strength gradient and the fourth-sensed magnetic field strength gradient for each array of the plurality.

8. The magnet detector of claim 7, wherein the processor compares the ambient gradient array value and the difference gradient array value, determines a deviation (Δ=|(Mᵥ(x,y,z)_{ambient})−(Mᵥ(x,y,z)_{anomaly})|) exceeding a threshold for the processor, and signals an output representative of the deviation as indicative of presence of the magnetic field anomaly at approximately the first spatial location and the second spatial location

9. The magnet detector of claim 8, further comprising:
an alarm connected to the processor, the output of the processor representative of the deviation sounds the alarm indicating the magnetic field anomaly at approximately the first spatial location and the second spatial location.

10. A hand-holdable probe incorporating the detector of claim 1.

11. A hand-holdable probe incorporating the detector of claim 4.

12. A hand-holdable wand incorporating the detector of claim 7.

13. A method of detecting presence of a magnetic tag of an item in a surgical patient, comprising the steps of:
first sensing a one-dimensional characteristic of a three-dimensional magnetic field at the patient at a spatial location;
first processing the one-dimensional characteristic from the step of first sensing, to obtain an ambient magnetic field scalar (Mᵥ(x)_{ambient}) for the spatial location;
second sensing the one-dimensional characteristic of the three-dimensional magnetic field at the patient at the spatial location, the magnetic tag present at about the spatial location;
second processing the one-dimensional characteristic of the step of second sensing, to obtain an anomolous magnetic field scalar (Mᵥ(x)a_{anomaly}) for the spatial location;
calculating a difference value (Δ=|(Mᵥ(x)_{ambient})−(Mᵥ(x)_{anomaly})|) in the ambient magnetic field scalar and the anomalous magnetic field scalar for the spatial location; and
signaling if the difference value exceeds a threshold level, indicative of presence of the magnetic tag of the device at about the spatial location.

14. The method of claim 13, further comprising the step of:
retrieving the device and the magnetic tag of the device.

15. The method of claim 14, wherein the signaling step effects an alarm selected from the group consisting of: audible sound, visual display, input-output, and combinations.

16. A method of detecting presence of a magnetic tag of an item in a surgical patient, comprising the steps of:
first sensing a two-dimensional characteristic of a three-dimensional magnetic field at the patient at a spatial location;
first processing the two-dimensional characteristic from the step of first sensing, to obtain an ambient magnetic field strength array (Mᵥ(x,y)_{ambient}) for the spatial location;
second sensing the two-dimensional characteristic of the three-dimensional magnetic field at the patient at the spatial location, the magnetic tag present at about the spatial location;
second processing the two-dimensional characteristic of the step of second sensing, to obtain an anomalous magnetic field strength array (Mᵥ(x,y)_{anomaly}) for the spatial location;
calculating a difference array value (Δ=|(Mᵥ(x,y)_{ambient})−(Mᵥ(x,y)_{anomaly})|) in the ambient magnetic field strength array and the anomalous magnetic field strength array for the spatial location; and
signaling if the difference array value exceeds a threshold level, indicative of presence of the magnetic tag of the device at about the spatial location.

17. The method of claim 16, further comprising the step of:
retrieving the device and the magnetic tag of the device.

18. The method of claim 17, wherein the step of signaling effects an alarm selected from the group consisting of: audible sound, visual display, input-output, and combinations.

19. A method of detecting presence of a magnetic tag of an item in a surgical patient, comprising the steps of:
first sensing a three-dimensional characteristic of a three-dimensional magnetic field at the patient at a spatial location;
first processing the three-dimensional characteristic from the step of first sensing, to obtain an ambient magnetic field gradient array (Mᵥ(x,y,z)_{ambient}) for the spatial location;
second sensing the three-dimensional characteristic of the three-dimensional magnetic field at the patient at the spatial location, the magnetic tag present at about the spatial location;
second processing the three-dimensional characteristic of the step of second sensing, to obtain an anomalous magnetic field gradient array (Mᵥ(x,y,z)_{anomaly}) for the spatial location;
calculating a difference gradient array value (Δ=|(Mᵥ(x,y,z)_{ambient})−(Mᵥ(x,y,z)_{anomaly})|) in the ambient magnetic field gradient array and the anomalous magnetic field gradient array for the spatial location; and
signaling if the difference gradient array value exceeds a threshold level, indicative of presence of the magnetic tag of the device at about the spatial location.

20. The method of claim 19, further comprising the step of:
retrieving the device and the magnetic tag of the device.

21. The method of claim 20, wherein the step of signaling effects an alarm selected from the group consisting of: audible sound, visual display, input-output, and combinations.

22. A method of detecting presence of a magnetic tag of an item in a surgical patient, comprising the steps of:
first moving a detector at a spatial location of an ambient magnetic field at the patient;
saving a characteristic of the ambient magnetic field at the spatial location;
second moving the detector in a magnetic anomaly to the ambient magnetic field, induced to the ambient magnetic field at the spatial location the patient; and
determining the presence of the magnetic anomaly at about the spatial location by the detector.

23. The method of claim 22, further comprising the step of:
signaling an alarm of the detector indicative of detected presence of the magnetic anomaly in the step of second moving.

24. The method of claim 23, further comprising the step of:
retrieving the magnetic tag and the item, if presence of the magnetic tag at about the spatial location is source of the magnetic anomaly.

25. A magnetic detector for locating a magnetically tagged item in a surgical patient, the item is contained within a patient body cavity located a distance along a one-dimensional axis in space from the magnetic detector, comprising:
a first sensor array in a first plane in space centered at and perpendicular to the one-dimensional; axis
a second sensor array positioned in a second plane in space, parallel to the first plane but not in the first plane, centered at and perpendicular to the one-dimensional axis;
a third sensor array positioned in a third plane in space, parallel to the first plane and second plane but not in either the first plane or the second plane, centered at and perpendicular to the one-dimensional axis;
wherein the magnetic detector has a three-dimensional volume of interest for detections, the three-dimensional volume extending in space along the one-dimensional axis generally into the body cavity in space, the three-dimensional volume being defined in space extending along the axis as centrum of the three-dimensional volume in direction of the axis, having an outer extent being defined as the greatest extension point in space from the axis that is outwardly extending for the first array in the first plane, the second array in the second plane, and the third array in the third plane, whichever is greatest outwardly extending in respective planes;
wherein the magnetic detector has a sensor volume of interest for detections, the sensor volume extending in space three-dimensionally along the one-dimensional axis within the three-dimensional volume, the sensor volume extending in direction of the axis having centrum along the axis, having an outer extent of the sensor volume being defined by a uniquely detectable region central to the three-dimensional volume for, collectively, the first array, the second array and the third array;
wherein the magnetic detector is capable of cancelling out any relevant ambient magnetic effects, automatically, because of the uniquely detectable region being formed by at least one, but not all, of the sensors of, collectively, the first array, second array and the third array, and the sensors not included within the uniquely detectable region being sufficient for magnetic vector gradient determination via comparison with sensors of the uniquely detectable region;
wherein the magnetic detector, when positioned at a location from the patient, finds the item at a vicinity of the patient body cavity centered at the axis extending into the patient, because of difference of magnetic vector gradient then-determined by the magnetic detector as being in excess of a threshold value, the threshold value being related to magnetic vector gradient for the same location as if without presence.

26. A detector for locating a magnetized tag in a human body, the magnetized tag is positioned in a direction (A) from the detector, comprising:
a first array of planarly arranged individual sensor elements having a first centrum located in the direction (A) with respect to the tag, of a first plane;
a second array of planarly arranged individual sensor elements having a second centrum located in the direction (A) with respect to the tag, of a second plane that is not the first plane;
wherein the second array is positioned, in space, parallel to the first array;
wherein an imaginary three-dimensional volume, extending longitudinally perpendicular to the first array and the second array and defined by extents of the first array and the second array in respective first plane and second plane, is an area of interest for detections by the detector;
wherein the detector effectively discounts, automatically, any relevant ambient magnetic effects substantially within the area of interest;
wherein the detector locates the tag in the human body when the tag is within the area of interest the tag, based on a difference value of magnetic vector gradient detected in presence of the tag versus magnetic vector gradient detected without presence of the tag, at about the same location for the area of interest, respectively.

27. The detector of claim 26, further comprising:
a third array of planarly arranged individual sensor elements having a third centrum located in the direction (A) with respect to the tag, of a third plane that is not the first plane and the second plane;
wherein the imaginary three-dimensional volume that is the area of interest is also defined by the extents of the third array in the third plane.
